(19) United States
(12) Patent Application Publication
(10) Pub. No.: US 2013/0266610 A1
(43) Pub. Date: Oct. 10, 2013

(54) STIMULATING ANTI-TUMOR IMMUNE RESPONSE AND BOOSTING CANCER IMMUNOTHERAPY VIA EXPRESSION OF 15-PGDH

(71) Applicants: Sergei A. Kusmartsev, Gainesville, FL (US); Sergey Kaliberov, Birmingham, AL (US); Evgeniy Eruslanov, Gainesville, FL (US); Johannes W. Vieweg, Gainesville, FL (US)

(72) Inventors: Sergei A. Kusmartsev, Gainesville, FL (US); Sergey Kaliberov, Birmingham, AL (US); Evgeniy Eruslanov, Gainesville, FL (US); Johannes W. Vieweg, Gainesville, FL (US)

(73) Assignee: University of Florida Research Foundation, Inc., Gaiesville, FL (US)

(21) Appl. No.: 13/854,517

(22) Filed: Apr. 1, 2013

Related U.S. Application Data
(62) Division of application No. 12/934,665, filed on Nov. 1, 2010.
(60) Provisional application No. 61/041,106, filed on Mar. 31, 2008.

(51) Int. Cl. A61K 39/00; A61K 38/44; A61K 48/00

(52) U.S. Cl. CPC A61K 39/0011; A61K 48/0058; A61K 38/443
USPC 424/199.1; 424/277.1

(57) ABSTRACT

Disclosed herein are methods and materials for inhibiting tumor growth by administering viral vectors to tumor cells. Particularly exemplified herein are methods of inhibiting tumor growth of colon tumors by delivering 15-PGDH to tumor environment. Antigen presenting cells may be coadministered with 15-PGDH.

### RELATED APPLICATIONS

This application is a divisional of U.S. application Ser. No. 12/934,665 filed Sep. 27, 2010, which is a national stage of PCT/US09/38946 filed Mar. 31, 2009, which claims priority to U.S. Ser. No. 61/041,106 filed Mar. 31, 2008, which are incorporated herein in their entirety.

Induction of tumor-specific immunity is an attractive approach for cancer therapy because of the prospect of harnessing the body's own defense mechanisms, rather than using standard toxic therapeutic agents, to provide long-term protection against tumor existence, growth and recurrence. This strategy is attractive for its potential to destroy small metastatic tumors which may escape detection, and to provide immunity against recurrent tumors.

In principle, an immunotherapy would depend on the presence of tumor-specific antigens and on the ability to induce a cytotoxic immune response that recognizes tumor cells which present antigens. Cytotoxic T lymphocytes (CTL) recognize major histocompatibility complex (MHC) class I molecules complexed to peptides derived from cellular proteins presented on the cell surface, in combination with co-stimulatory molecules. Mueller et al., Annu. Rev. Immunol. 7: 445-80 (1989). In fact, tumor-specific antigens have been detected in a range of human tumors. Roth et al., Adv. Immunol. 57: 281-351 (1994); Boon et al., Annu. Rev. Immunol. 12: 337-65 (1994).

Some cancer vaccination strategies have focused on the use of killed tumor cells or lysates delivered in combination with adjuvants or cytokines. More recently, gene transfer of cytokines, MHC molecules, co-stimulatory molecules, or tumor antigens to tumor cells has been used to enhance the tumor cell's visibility to immune effector cells. Dranoff & Mulligan, Adv. Immunol. 58: 417-54 (1995).

The therapeutic use of “cancer vaccines” has presented major difficulties, however. In particular, conventional approaches require obtaining and culturing a patient's autologous tumor cells for manipulation in vitro, irradiation and subsequent vaccination, or the identification and purification of a specific tumor antigen.

A number of transfection systems have been developed to deliver heterologous genes into in vivo tumors to investigate cancer gene therapy, but all have limitations.

### BRIEF DESCRIPTION OF THE DRAWINGS

FIG. 1. (A) In vitro transduction of tumor cells with Ad-PGDH results in 15-PGDH protein expression. Expression of 15-PGDH protein in tumor cell lysates was evaluated using Western blotting. (B, C) In vivo intra-tumoral delivery of PGDH gene in tumor-bearing mice promotes inhibition of tumor growth. CT-26 murine colon carcinoma cells (5×105) were inoculated subcutaneously in BALB/c mice. When tumor size reached 20-25 mm2 size, mice were randomly divided in two groups. First group of tumor-bearing mice was injected i.t. with control adenovirus (C, left panel) and another group with adenovirus encoding 15-PGDH (C, right panel). Kinetic of tumor growth in individual mouse is shown.

FIG. 2: PGDH gene delivery changes cytokine profile of intra-tumoral myeloid cells CT-26 murine colon carcinoma cells 5×105 CT26 tumor cells were inoculated subcutaneously in BALB/c mice. When tumors reached 20-25 mm2 size (day 7), mice were randomly divided in three groups. First group of tumor-bearing mice was injected with PBS (control), second group—with control adenovirus (control Ad), and third group with adenovirus encoding 15-PGDH (Ad-PGDH). Next day after last injection of adenovirus mice were sacrificed; tumors were excised and intra-tumoral CD11b cell were isolated with magnetic beads. CD11b cells were plated in 6-well cell culture plates and cultured in humidified CO2 incubator at 37° C. 24 h later cell supernatants were collected, filtered and assayed for presence of PGE2 and cytokines by ELISA and Multiplex assay, respectively. Collected CD11b cells were used for analysis of intracellular cytokine expression with flow cytometry. (A, left panel), PGE2 production by intra-tumoral CD11b cells. Concentration of PGE2 determined in CD11b cell supernatants by ELISA; (A, right panel), Number of intratumoral CD11b cells in Ad-PGDH-treated mice is increased; Average mean±SD are shown.
(B) Expression of cytokines by tumor-infiltrated CD11b cells derived from Ad-PGDH treated and control animals. CD11b cells were stained first for surface marker F4/80, then for intracellular IL-10, IL-6, TNF-alpha or IL-12 as described in Materials and Methods, and then analyzed by flow cytometry. Results of one representative experiment are shown. (C) Cytokines secretion by tumor-infiltrated CD11b cells. Concentration of IL-1beta, eotaxin and RANTES was measured using Multiplex assay. Average mean±SD are shown

FIG. 3. Delivery of PGDH gene promotes in situ APC differentiation/maturation. (A). PGE₂ inhibits GM-CSF-driven differentiation of myeloid CD11c dendritic cells. Bone marrow cells from naïve mice were cultured in presence of recombinant GM-CSF (20 ng/ml). Exogenous PGE₂ at two different concentrations was added to the cultures at the time of cell culture initiation. Seven days later cells were collected, washed, stained for CD11c and F4/80 and analyzed by flow cytometry. (B). Administration of Ad-PGDH promotes in situ differentiation of intra-tumoral antigen-presenting cells. Tumor-bearing mice were treated with Ad-PGDH as described. The next day after the last Ad injection mice were sacrificed. Tumors from treated and control animals were dissected, digested with collagenase cocktail and then CD11b cells were isolated with magnetic beads. CD11b cells were cultured for 24 hours in complete culture medium; cells were collected, stained for CD11c and F4/80 and analyzed by flow cytometry.
(C) Tumors from treated and control animals were dissected, digested with collagenase cocktail and then CD11b cells were isolated with magnetic beads. CD11b cells were cultured for 24 hours in complete culture medium; cells were collected, stained for CD11c and F4/80 and analyzed by flow cytometry. Representative experiment is shown.

FIG. 4: Introduction of the 15-PGDH gene in tumor microenvironment reverses the immunosuppressive profile of tumor-infiltrated CD11b cells. (A) Ad-PGDH-mediated treatment results in the inhibition of IL-13 production by tumor-infiltrated CD11b cells. CT-26 tumor bearing mice were established and treated with Ad-PGDH or control Ad as described in FIG. 2. 48 hours after last injection of Ad-PGDH or control virus (control Ad) mice were sacrificed, and intratumoral CD11b cells were isolated with magnetic beads. CD11b cells were added in 6-well plates (1×10⁶ cells/ ml). After 24 hours of incubation, cell supernatants were collected and the concentration of IL-13 was measured by Multiplex assay. Average±SD are shown. (B) Arginase activity. Whole cell lysates were prepared from intra-tumoral CD11b cells derived from Ad-PGDH-treated or control tumor-bearing mice. Arginase activity was measured spectrophotometrically as described in Materials and Methods. (C) Expression of arginases I and II in CD11b cells. Samples (30 μg of protein) were subjected to electrophoresis in 10% SDS-polyacrylamide gels, blotted onto 0.45-μm nitrocellulose membranes and probed with anti-arginase I or II antibody. (D) Expression of phosphorylated and total STATE in tumor-infiltrated CD11b cells was evaluated by Western blotting.

FIG. 5. (A). Survival curve. CT-26 tumor cells were injected s.c. into BALB/c mice. Once tumors were established (day 7) mice were randomly divided in four groups: 1) control (untreated); 2) DC plus control Ad; 3) Ad-PGDH alone; 4) DC+Ad-PGDH. Each group consisted of five mice. Adenovirus was injected intratumorally (2×108 TCID50) twice a week starting on day 7 after tumor inoculation (five injections in total). Bone marrowderived DCs were injected into tumor site three times starting on day 10 with five days interval. Percentage of survived animals over time is shown.
(B) IFN-gamma by lymph node cells in response in vitro with irradiated CT-26 or 4T1 murine tumors. Concentration of cytokines was determined using Multiplex assay.
Experimental design was similar to described above in FIG. 2B. Average±SD are shown.

FIG. 6. Catabolism of PGE2 in tumor-infiltrated CD11b cells, derived from murine colon carcinoma, is altered. CT-26 tumor cells were injected s.c. into BALB/c mice. On day fourteen after tumor injection, tumors harvested and digested with collagenase cocktail. Intra-tumoral CD11b cells were isolated with magnetic beads. PGE2 production (A), was measured in cell supernatant after 24 incubation by ELISA. COX-2 (B), mPGES1 (C) and 15-PGDH (D) gene expression was determined in freshly isolated intra-tumoral CD11b cells by qRT-PCR.

FIG. 7. In vivo fla promoter activity. BALB/c mice were injected with 2×10⁵ CT-26 tumor cells. On day 10 after tumor cell inoculation 2×10⁸ TCID50 of adenovirus encoding Renilla luciferase gene under flt1 promoter was administered into tumor site. Forty-eight hours later, mice were sacrificed and excised tumors were digested with collagenase cocktail. Luciferase activity was determined in cell lysates obtained from whole tumor cell population, isolated tumor-infiltrated CD11b cells and in CD11b-negative tumor Average±SD are shown. Luciferase activity values were normalized to Renilla luciferase activity.

FIG. 8. PGDH gene delivery inhibits PGE2 secretion and changes cytokine profile in intra-tumoral CD11b myeloid cells. Tumor cells were inoculated s.c in mice at day 0. When tumors reached 20-25 mm² size (day seven), mice were randomly divided in three groups. The first group of tumor-bearing mice was injected with PBS (control), second group—with control adenovirus (control Ad), and third group with adenovirus encoding 15-PGDH (Ad-PGDH, twice with three day interval). The following day, after the last injection of Ad, mice were sacrificed; tumors were excised and intra-tumoral CD11b cells were isolated with magnetic beads (Left panel). 1×10⁶ CD11b cells were plated in 6-well cell plates and cultured for 24 h. Then cell supernatants were collected, filtered and assayed for presence of PGE₂ by ELISA (central panel). Collected CD11b cells were lysed and expression of COX-2 and mPGES1 protein was measured by Western blotting (right panel).

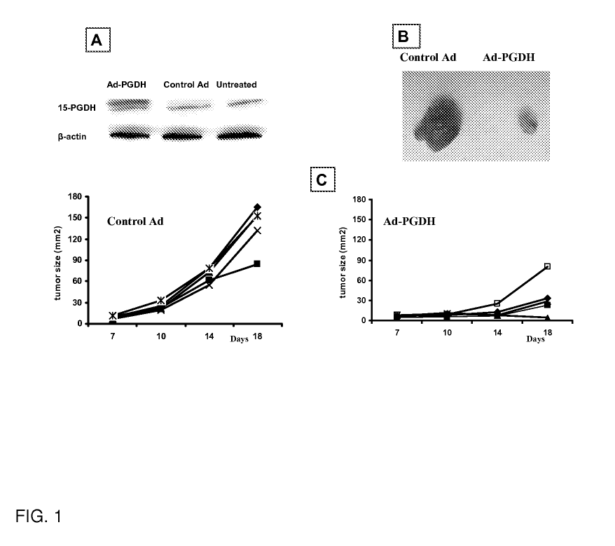

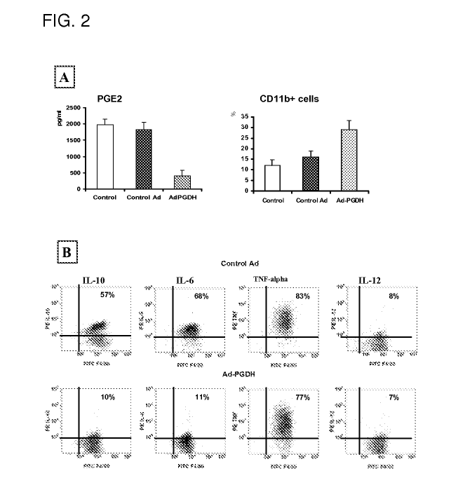

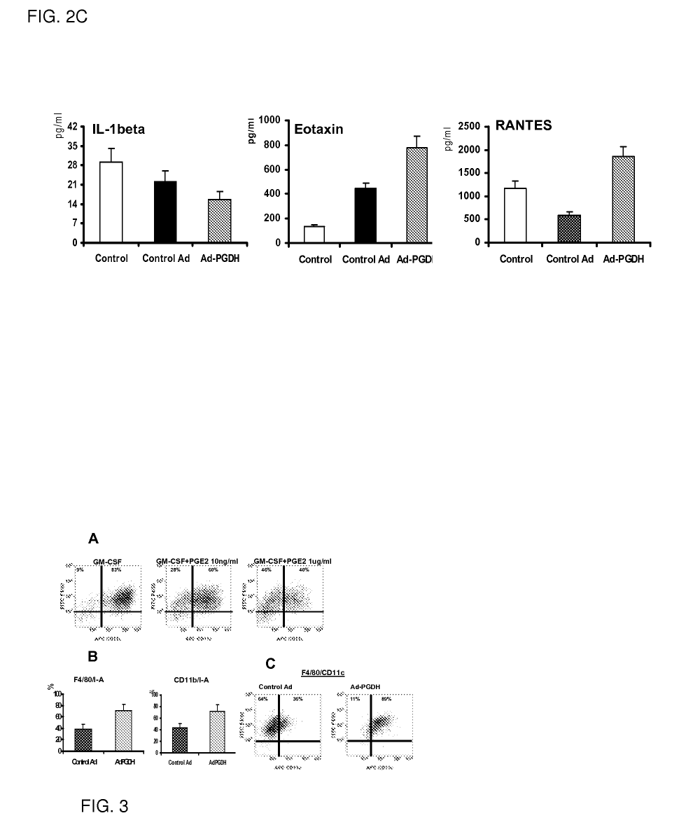

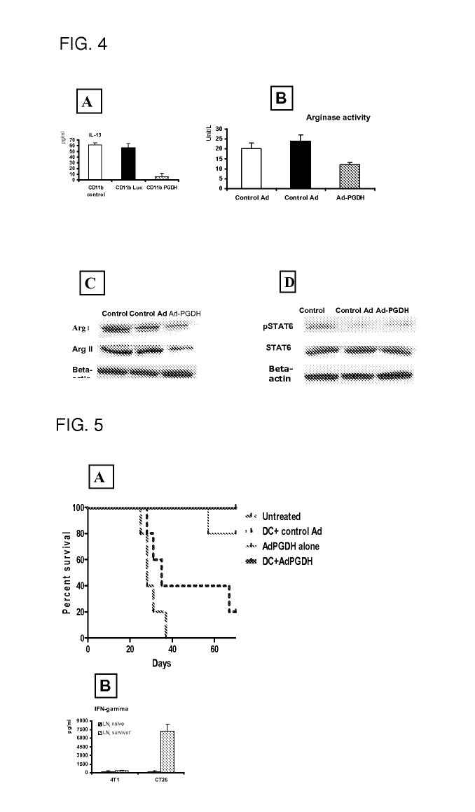

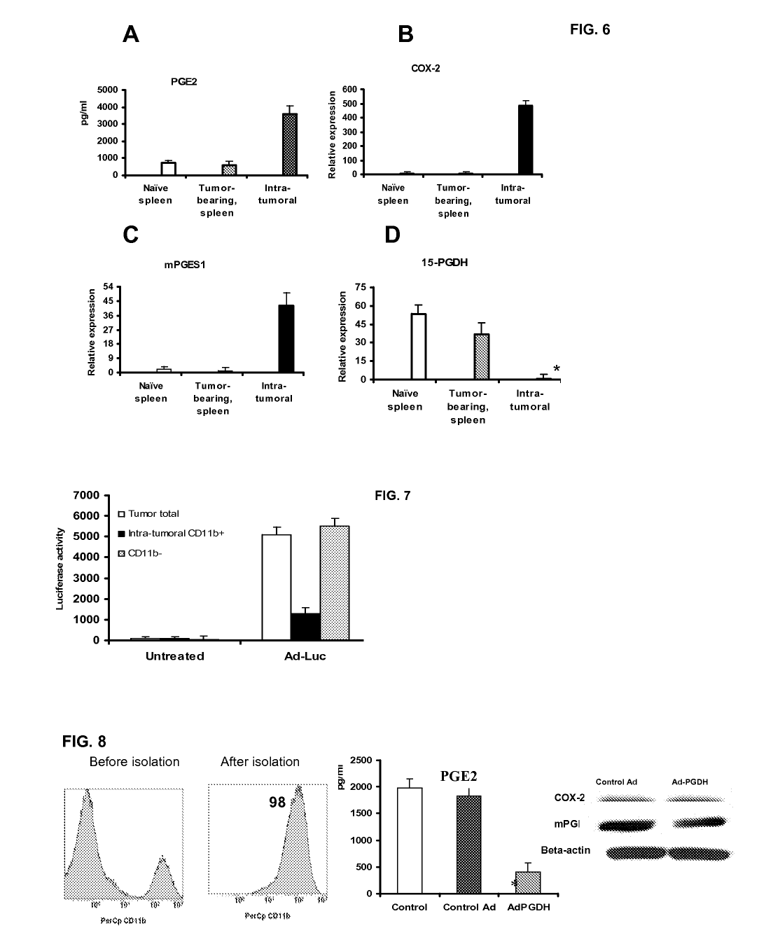

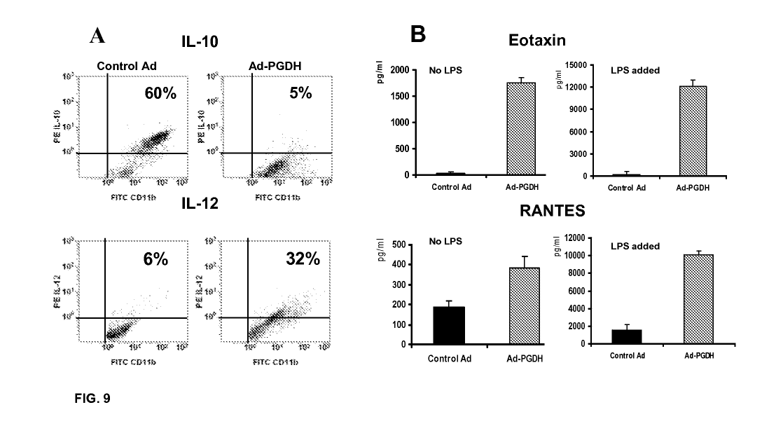

FIG. 9. Introduction of the 15-PGDH gene in tumor microenvironment promotes switch of cytokine profile in draining lymph nodes. CT-26 tumor bearing mice were established and treated with Ad-PGDH as described in FIG. 1. Draining lymph nodes were isolated next day after the last injection of Ad-PGDH or control Ad. Lymph node-derived cell suspension was plated in 6-well plates (1×10⁶ cells/ml). After 24 hours of incubation, cells were collected and analyzed by flow cytometry for intracellular cytokines (IL-10 and IL-12) (A). Concentration of cytokines in cell supernatants was measured using Multiplex assay (B). Average mean±SD is shown. Results of one representative experiment out of two are shown.

### DETAILED DESCRIPTION

The invention is based on the inventors work relating to tumor inhibition by manipulating the microenvironment of the tumor by the forced expression of certain proteins. In particular, the inventors show that the delivery of NAD-dependent 15-prostaglandin dehydrogenase (15-PGDH) gene in mice with established prostate or colon tumors results in substantial suppression of in vivo tumor growth. Importantly, this anti-tumor therapeutic effect of 15-PGDH gene delivery was associated with dramatic inhibition of production PGE2 and protumoral Th2 cytokines (IL-10, IL-6) by tumor-infiltrated myeloid cells as well as markedly improved differentiation of antigen-presenting cells. The inventors also examined whether the combination of 15-PGDH gene therapy with dendritic cell (DC)-based immunotherapy may have synergistic therapeutic effect. Obtained results indicated that combined treatment of mice with pre-established CT-26 colon carcinoma tumors with 15-PGDH and DC induces complete tumor eradication. All survived mice were still alive after 70 days. This data show that conditioning the tumor microenvironment with 15-PDGH results in correction of tumor-induced immune dysfunction and synergistically boost therapeutic effect of cancer immunotherapy.

FIG. 6A shows that isolated intra-tumoral CD11b cells secrete substantial amounts of PGE₂ PGE2 production by tumor-infiltrated CD11b cells was associated with high expression of major PGE2-forming enzymes in these cells: COX-2 and mPGES1 (FIGS. 6B and C). On the other hand, expression of major PGE-catabolizing enzyme 15-PGDH in tumor-infiltrated CD11b myeloid cells was substantially down-regulated (FIG. 6D).

In one embodiment, the invention is directed to a method of suppressing tumor growth in a subject. The method includes delivering a 15-PGDH gene to the subject via a mechanism for causing the expression of such gene in vivo. Gene delivery may take the form of naked DNA, or vectors (such as viral vectors) containing expression constructs configured to express protein in or proximate to the tumor. Typically, the gene is delivered via injection into and/or proximate to the tumor (i.e. administered in such a way that vector is exposed to tumor cells or environment surrounding cell(s) or adjacent to tumor cells, i.e., tumor microenvironment). In a specific embodiment, the gene is delivered via an adenoviral vector containing the 15-PGDH gene. Alternatively, the gene is delivered in the subject such that either the gene or the gene product is transported to the tumor.

According to another embodiment, the invention is directed to a method of treating a tumor in a subject in need thereof, the method comprising the coadministration of a 15-PGDH gene and Antigen Presenting Cells (APCs, e.g., dendritic cells). Coadministration, as used herein, means that delivered gene (or expressed gene product) and delivered dendritic cells are present in the subject at the same time, or within up to a week of each other. Dendritic cells may be isolated from the subject or an allogeneic source. Further, dendritic cells may be activated against tumor antigens ex vivo, before administration to the subject.

In another embodiment, the invention pertains to a viral vector comprising a polynucleotide encoding 15-PGDH. In a specific embodiment, the viral vector is an adenoviral vector. A related embodiment pertains to a DNA plasmid comprising a nucleic acid sequence encoding 15-PGDH.

As used herein, antigen presenting cells include but are not limited to dendritic cells, macrophages or natural killer cells. Other examples of cells that could serve as antigen presenting cells, include fibroblasts, glial cells and microglial cells.

### Polynucleotides and Polypeptides

Polynucleotides and polypeptides having substantial identity to the nucleotide and amino acid sequences relating to PGDH (SEQ ID NOS. 1-4) used in conjunction with present invention can also be employed in preferred embodiments. Here “substantial identity” means that two sequences, when optimally aligned such as by the programs GAP or BESTFIT (peptides) using default gap weights, or as measured by computer algorithms BLASTX or BLASTP, share at least 50%, preferably 75%, and most preferably 95% sequence identity. Preferably, residue positions which are not identical differ by conservative amino acid substitutions. For example, the substitution of amino acids having similar chemical properties such as charge or polarity are not likely to effect the properties of a protein. Non-limiting examples include glutamine for asparagine or glutamic acid for aspartic acid.

The term “variant” as used herein refers to nucleotide and polypeptide sequences wherein the nucleotide or amino acid sequence exhibits substantial identity with the nucleotide or amino acid sequence of SEQ ID NOs 1 & 2, preferably 75% sequence identity and most preferably 90-95% sequence identity to the sequences of the present invention: provided said variant has a biological activity as defined herein. The variant may be arrived at by modification of the native nucleotide or amino acid sequence by such modifications as insertion, substitution or deletion of one or more nucleotides or amino acids or it may be a naturally occurring variant. The term “variant” also includes homologous sequences which hybridise to the sequences of the invention under standard or preferably stringent hybridisation conditions familiar to those skilled in the art. Examples of the in situ hybridisation procedure typically used are described in (Tisdall et al., 1999); (Juengel et al., 2000). Where such a variant is desired, the nucleotide sequence of the native DNA is altered appropriately. This alteration can be made through elective synthesis of the DNA or by modification of the native DNA by, for example, site-specific or cassette mutagenesis. Preferably, where portions of cDNA or genomic DNA require sequence modifications, site-specific primer directed mutagenesis is employed, using techniques standard in the art.

In specific embodiments, a variant of a polypeptide is one having at least about 80% amino acid sequence identity with the amino acid sequence of a native sequence full length sequence of the plant degrading enzymes provided on the attached 10669-034SEDID ASCII file. Such variant polypeptides include, for instance, polypeptides wherein one or more amino acid residues are added, or deleted, at the N- and/or C-terminus, as well as within one or more internal domains, of the full-length amino acid sequence. Fragments of the peptides are also contemplated. Ordinarily, a variant polypeptide will have at least about 80% amino acid sequence identity, more preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and yet more preferably at least about 99% amino acid sequence identity with a polypeptide encoded by a nucleic acid molecule or a specified fragment thereof. Ordinarily, variant polypeptides are at least about 10 amino acids in length, often at least about 20 amino acids in length, more often at least about 30 amino acids in length, more often at least about 40 amino acids in length, more often at least about 50 amino acids in length, more often at least about 60 amino acids in length, more often at least about 70 amino acids in length, more often at least about 80 amino acids in length, more often at least about 90 amino acids in length, more often at least about 100 amino acids in length, or more.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to re-anneal when complementary strands are present in an environment below their melting temperature. The higher the degree of desired identity between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, are identified by those that: (1) employ low ionic strength and high temperature for washing, 0.015 M sodium chloride/0.0015 M sodium citrate/0.1% sodium dodecyl sulfate at 500 C.; (2) employ during hybridization a denaturing agent, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42 degrees C.; or (3) employ 50% formamide, 5×SSC (0.75 M NaCl, 0.075 M sodium citrate), 50 mM sodium phosphate (pH 6.8), 0.1% sodium pyrophosphate, 5.times. Denhardt's solution, sonicated salmon sperm DNA (50 μg/ml), 0.1% SDS, and 10% dextran sulfate at 42 degrees C., with washes at 42 degrees C. in 0.2×SSC (sodium chloride/sodium citrate) and 50% formamide at 55 degrees C., followed by a high-stringency wash consisting of 0.1×SSC containing EDTA at 55 degrees C.

“Moderately stringent conditions” are identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50 degrees C. The skilled artisan will recognize how to adjust the temperature, ionic strength, etc. as necessary to accommodate factors such as probe length and the like.

Typically, for stringent hybridization conditions a combination of temperature and salt concentration should be chosen that is approximately 12-20° C. below the calculated Tₘ of the hybrid under study. The Tₘ of a hybrid between an polynucleotide having a nucleotide sequence shown in SEQ ID NO: 1 or the complement thereof and a polynucleotide sequence which is at least about 50, preferably about 75, 90, 96, or 98% identical to one of those nucleotide sequences can be calculated, for example, using the equation of Bolton and McCarthy, Proc. Natl. Acad. Sci. U.S.A. 48, 1390 (1962):

Tₘ=81.5° C.−16.6(log₁₀ [Na⁺])+0.41(% G+C)−0.63(% formamide)−600/l),

where l=the length of the hybrid in basepairs.

In a specific embodiment, stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C.

### Vectors

In some embodiments, vectors adenovirus -based vectors are used to transfect cells with 15-PGDH, in particular, replication defective adenovirus-based vectors. Other vectors of the invention used in vitro, in vivo, and ex vivo include viral vectors, such as retroviruses (including lentiviruses), herpes viruses, alphavirus, adeno-associated viruses, vaccinia virus, papillomavirus, or Epstein Barr virus (EBV).

Methods for constructing and using viral vectors are known in the art (see, e.g., Miller and Rosman, BioTechniques 1992, 7:980-990). In accordance with the present invention there may be employed conventional molecular biology, microbiology, and recombinant DNA techniques within the skill of the art. Such techniques are well-known and are explained fully in the literature. See, e.g., Sambrook, Fritsch and Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

Various companies produce viral vectors commercially, including but by no means limited to Avigen, Inc. (Alameda, Calif.; AAV vectors), Cell Genesys (Foster City, Calif.; retroviral, adenoviral, AAV vectors, and lentiviral vectors), Clontech (retroviral and baculoviral vectors), Genovo, Inc. (Sharon Hill, Pa.; adenoviral and AAV vectors), Genvec (adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), and Transgene (Strasbourg, France; adenoviral, vaccinia, retroviral, and lentiviral vectors).

In certain embodiments, the viral vectors of the invention are replication defective, that is, they are unable to replicate autonomously in the target cell. Preferably, the replication defective virus is a minimal virus, i.e., it retains only the sequences of its genome which are necessary for target cell recognition and encapsidating the viral genome. Replication defective virus is not infective after introduction into a cell. Use of replication defective viral vectors allows for administration to cells in a specific, localized area, without concern that the vector can infect other cells. Thus, a specific tissue can be specifically targeted. Examples of particular vectors include, but are not limited to, defective herpes virus vectors (see, e.g., Kaplitt et al., Molec. Cell. Neurosci. 1991, 2:320-330; Patent Publication RD 371005 A; PCT Publications No. WO 94/21807 and WO 92/05263), defective adenovirus vectors (see, e.g., Stratford-Perricaudet et al., J. Clin. Invest. 1992, 90:626-630; La Salle et al., Science 1993, 259:988-990; PCT Publications No. WO 94/26914, WO 95/02697, WO 94/28938, WO 94/28152, WO 94/12649, WO 95/02697, and WO 96/22378), and defective adeno-associated virus vectors (Samulski et al., J. Virol. 1987, 61:3096-3101; Samulski et al., J. Virol. 1989, 63:3822-3828; Lebkowski et al., Mol. Cell. Biol. 1988, 8:3988-3996; PCT Publications No. WO 91/18088 and WO 93/09239; U.S. Pat. Nos. 4,797,368 and 5,139,941; European Publication No. EP 488 528).

Adenovirus-based vectors. Adenoviruses are eukaryotic DNA viruses that can be modified to efficiently deliver a nucleic acid of the invention to a variety of cell types. Various serotypes of adenovirus exist. Of these serotypes, preference is given, within the scope of the present invention, to using type 2 or type 5 human adenoviruses (Ad 2 or Ad 5) or adenoviruses of animal origin (see PCT Publication No. WO94/26914). Those adenoviruses of animal origin which can be used within the scope of the present invention include adenoviruses of canine, bovine, murine (e.g., Mav1 [Beard et al., Virology, 1990, 75:81]), ovine, porcine, avian, and simian (e.g., SAV) origin. Preferably, the adenovirus of animal origin is a canine adenovirus, more preferably a CAV2 adenovirus (e.g., Manhattan or A26/61 strain [ATCC Accession No. VR-800]). Various replication defective adenovirus and minimum adenovirus vectors have been described (PCT Publications No. WO94/26914, WO95/02697, WO94/28938, WO94/28152, WO94/12649, WO95/02697, WO96/22378). The replication defective recombinant adenoviruses according to the invention can be prepared by any technique known to the person skilled in the art (Levrero et al., Gene, 1991, 101:195; EP Publication No. 185 573; Graham, EMBO J., 1984, 3:2917; Graham et al., J. Gen. Virol., 1977, 36:59). Recombinant adenoviruses are recovered and purified using standard molecular biological techniques, which are well known to one of ordinary skill in the art.

Adeno-associated virus-based vectors. The adeno-associated viruses (AAV) are DNA viruses of relatively small size which can integrate, in a stable and site-specific manner, into the genome of the cells which they infect. They are able to infect a wide spectrum of cells without inducing any effects on cellular growth, morphology or differentiation, and they do not appear to be involved in human pathologies. The AAV genome has been cloned, sequenced and characterized. The use of vectors derived from the AAVs for transferring genes in vitro and in vivo has been described (see PCT Publications No. WO 91/18088 and WO 93/09239; U.S. Pat. Nos. 4,797,368 and 5,139,941; EP Publication No. 488 528). The replication defective recombinant AAVs according to the invention can be prepared by cotransfecting a plasmid containing the nucleic acid sequence of interest flanked by two AAV inverted terminal repeat (ITR) regions, and a plasmid carrying the AAV encapsidation genes (rep and cap genes), into a cell line which is infected with a human helper virus (e.g., an adenovirus). The AAV recombinants which are produced are then purified by standard techniques.

Retroviral vectors. In another embodiment, the invention provides retroviral vectors, e.g., as described in Mann et al., Cell 1983, 33:153; U.S. Pat. Nos. 4,650,764, 4,980,289, 5,124,263, and 5,399,346; Markowitz et al., J. Virol. 1988, 62:1120; EP Publications No. 453 242 and 178 220; Bernstein et al. Genet. Eng. 1985, 7:235; McCormick, BioTechnology 1985, 3:689; and Kuo et al., 1993, Blood, 82:845. The retroviruses are integrating viruses which infect dividing cells. The retrovirus genome includes two LTRs, an encapsidation sequence and three coding regions (gag, pol and env). Replication defective non-infectious retroviral vectors are manipulated to destroy the viral packaging signal, but retain the structural genes required to package the co-introduced virus engineered to contain the heterologous gene and the packaging signals. Thus, in recombinant replication defective retroviral vectors, the gag, pol and env genes are generally deleted, in whole or in part, and replaced with a heterologous nucleic acid sequence of interest. These vectors can be constructed from different types of retroviruses, such as HIV (human immuno-deficiency virus), MoMuLV (murine Moloney leukaemia virus), MSV (murine Moloney sarcoma virus), HaSV (Harvey sarcoma virus), SNV (spleen necrosis virus), RSV (Rous sarcoma virus), and Friend virus. Suitable packaging cell lines have been described in the prior art, in particular, the cell line PA317 (U.S. Pat. No. 4,861,719); the PsiCRIP cell line (PCT Publication No. WO 90/02806) and the GP+envAm-12 cell line (PCT Publication No. WO 89/07150). In addition, recombinant retroviral vectors can contain modifications within the LTRs for suppressing transcriptional activity as well as extensive encapsidation sequences which may include a part of the gag gene (Bender et al., J. Virol. 1987, 61:1639). Recombinant retroviral vectors are purified by standard techniques known to those having ordinary skill in the art.

Retrovirus vectors can also be introduced by DNA viruses, which permits one cycle of retroviral replication and amplifies transfection efficiency (see PCT Publications No. WO 95/22617, WO 95/26411, WO 96/39036, WO 97/19182).

In a specific embodiment of the invention, lentiviral vectors can be used as agents for the direct delivery and sustained expression of a transgene in several tissue types, including brain, retina, muscle, liver, and blood. This subtype of retroviral vectors can efficiently transduce dividing and nondividing cells in these tissues, and maintain long-term expression of the gene of interest (for a review, see, Naldini, Curr. Opin. Biotechnol. 1998, 9:457-63; Zufferey, et al., J. Virol. 1998, 72:9873-80). Lentiviral packaging cell lines are available and known generally in the art (see, e.g., Kafri, et al., J. Virol., 1999, 73: 576-584).

Non-viral vectors. In another embodiment, the invention provides non-viral vectors that can be introduced in vivo, provided that these vectors contain a targeting peptide, protein, antibody, etc. that specifically binds HALR. For example, compositions of synthetic cationic lipids, which can be used to prepare liposomes for in vivo transfection of a vector carrying an anti-tumor therapeutic gene, are described in Felgner et. al., Proc. Natl. Acad. Sci. USA 1987, 84:7413-7417; Felgner and Ringold, Science 1989, 337:387-388; Mackey, et al., Proc. Natl. Acad. Sci. USA 1988, 85:8027-8031; and Ulmer et al, Science 1993, 259:1745-1748. Useful lipid compounds and compositions for transfer of nucleic acids are described, e.g., in PCT Publications No. WO 95/18863 and WO96/17823, and in U.S. Pat. No. 5,459,127. Targeting peptides, e.g., laminin or HALR-binding laminin peptides, and proteins such as anti-HALR antibodies, or non-peptide molecules can be coupled to liposomes covalently (e.g., by conjugation of the peptide to a phospholipid or cholesterol; see also Mackey et al., supra) or non-covalently (e.g., by insertion via a membrane binding domain or moiety into the bilayer membrane).

Alphaviruses are well known in the art, and include without limitation Equine Encephalitis viruses, Semliki Forest virus and related species, Sindbis virus, and recombinant or ungrouped species (see Strauss and Strauss, Microbiol. Rev. 1994, 58:491-562, Table 1, p. 493).

As used herein the term “replication deficient virus” has its ordinary meaning, i.e., a virus that is propagation incompetent as a result of modifications to its genome. Thus, once such recombinant virus infects a cell, the only course it can follow is to express any viral and heterologous protein contained in its genome. In a specific embodiment, the replication defective vectors of the invention may contain genes encoding nonstructural proteins, and are self-sufficient for RNA transcription and gene expression. However, these vectors lack genes encoding structural proteins, so that a helper genome is needed to allow them to be packaged into infectious particles. In addition to providing therapeutically safe vectors, the removal of the structural proteins increases the capacity of these vectors to incorporate more than 6 kb of heterologous sequences. In another embodiment, propagation incompetence of the adenovirus vectors of the invention is achieved indirectly, e.g., by removing the packaging signal which allows the structural proteins to be packaged in virions being released from the packaging cell line.

### EXAMPLES

Recently, NAD-dependent 15-prostaglandin dehydrogenase (15-PGDH) has been identified as a tumor suppressor gene for several cancers including colon, breast and lung cancers. By inactivating endogenous prostaglandin E2 (PGE2), 15-PGDH provides an important, natural way to reduce this immunosuppressive and pro-carcinogenic lipid mediator. Previous studies have also demonstrated that tumors frequently overexpress COX-2 but at the same time inhibit expression and catabolic activity of 15-PGDH. In this example, we investigated whether restoration of 15-PGDH expression in tumor site could enhance catabolism of PGE2 and improve function of immune system. Our results demonstrate that adenovirus-mediated intratumoral delivery of 15-PGDH gene alone in mice with established prostate or colon tumors results in substantial inhibition of tumor growth, whereas an administration of control adenovirus did not affect tumor growth Importantly, that 15-PGDH-mediated anti-tumor effect was associated with dramatic inhibition of production immunosuppressive lipid mediator PGE2 and protumoral Th2 cytokines by tumor-infiltrated myeloid cells, and additionally, with markedly improved differentiation of antigen-presenting cells. The inventors next examined whether combination of 15-PGDH gene therapy with dendritic cell (DC)-based immunotherapy may have synergistic therapeutic effect. Obtained results indicate that combined treatment of mice with pre-established CT-26 colon carcinoma tumors with 15-PGDH and DC induces complete tumor eradication. All survived mice were still alive after 60 days. These data support the idea that conditioning of tumor microenvironment with 15-PGDH results in correction of tumor-induced immune dysfunction and may synergistically boost a therapeutic effect of cancer immunotherapy.

Female BALB/c mice (6-8 weeks of age) were obtained from the National Cancer Institute (Frederick, Md.). CT-26 murine colon carcinoma cell line was purchased from ATCC (Manassas, Va.). Replication-deficient E1- and E3-deleted adenoviral recombinant vectors encoding the human hpgd gene (Ad-PGDH) and control adenovirus encoding luciferase gene under the same promoter (control Ad) were constructed by using pAdEasy system (Quantum Biotechnologies, Montreal, Canada). To evaluate in vivo effect of 15-PGDH gene delivery, mice with pre-established tumors (16-25 mm2) were intra-tumorally (i.t) injected with 2×108 TCID50 of adenovirus encoding 15-PGDH or control adenovirus twice a week. Mice were sacrificed at indicated time.

### Example 1

### 15-PGDH Gene Delivery in Tumor Microenvironment Promotes Inhibition of Established Colon Cancer In Vivo Growth

To evaluate the in vivo anti-tumor effect of Ad-PGDH, the inventors established CT-26 colon tumors in mice through subcutaneous inoculation. Starting on day ten, when tumor size reached 16-25 mm², mice were given four intra-tumoral injections of adenovirus encoding 15-PGDH (Ad-PGDH) or control adenovirus (control Ad). As shown in FIG. 1, adenovirus-mediated delivery of the 15-PGDH gene in tumor tissue resulted in significant inhibition of tumor growth.

### Example 2

### Introduction of PGDH Gene in Tumor Microenvironment Reduces PGE2 and Th2 Cytokines Production

High expression of COX-2 and enhanced secretion of PGE2 by tumor cells is one of the most recognized mechanisms of immune deviation in cancer. The PGE2 overproduction has a major impact on intra-tumoral immune as well as inflammatory cells favoring Th2 cytokine milieu and promotoing immunosuppressive microenvironment. The inventors examined whether in vivo transfer of 15-PGDH gene may reduce PGE2 and cytokine secretion by intratumoral myeloid cells. We purified intra-tumoral CD11b cells from mice treated with Ad-PGDH or control virus and cultured them for 24 h. Then cell supernatants were collected and assayed for presence of PGE2 by ELISA (FIG. 2A). In addition, intracellular cytokine production was measured (FIG. 2B). Obtained results clearly indicated that intra-tumoral delivery of 15-PGDH gene significantly inhibits PGE2 secretion by tumor-associated myeloid cells (1800 ng/ml in control vs. 405 ng/ml in Ad-PGDH treated group). Importantly, this inhibition was associated with significant reduction of expression of pro-tumoral Th2 cytokines, such as IL-10 (57% in control vs. 10% in Ad-PGDH-treated mice) and IL-6 (68% in control vs. 11% in Ad-PGDH treated mice see FIG. 2B). Furthermore, 15-PGDH gene delivery promoted in situ differentiation of antigen-presenting cells, leading to up-regulation of MHC class II molecule and increased numbers of CD11c-positive dendritic cells in tmor site as well as draining lymph nodes (data not shown). Together, the data indicate condition of tumor microenvironment with 15-PGDH gene may correct anti-tumor responses. FIG. 2C shows cytokines secretion by tumor-infiltrated CD11b cells. Concentration of IL-1beta, eotaxin and RANTES was measured using Multiplex assay. The Ad-PGDH-treated mice showed lower expression of IL-1beta but higher expression of Eotaxin and RANTES compared to control.

### Example 3

### 15-PGDH Induces Differentiation of Tumor-Infiltrated Myeloid Antigen-Presenting Cells

PGE₂ is one of the main tumor-secreted factors responsible for altered APC differentiation in the tumor microenvironment. We tested, in vitro, whether the presence of PGE₂ could inhibit GM-CSF-driven APC differentiation from bone marrow progenitor cells. FIG. 3A demonstrates that the addition of PGE₂ to the cell cultures of normal bone marrow myeloid cell progenitors substantially reduces the number of CD11c-positive dendritic cells in a dose-dependent manner (83% in control vs. 40% in presence of 1 μg/ml PGE₂). The inventors wondered whether conditioning of the tumor microenvironment with the 15-PGDH gene could influence the in situ differentiation/maturation of intra-tumoral antigen-presenting cells (APC). First, we measured the expression of the MHCclass II molecule in tumor-infiltrated F4/80 and CD11b cells. The majority of intratumoral CD11b cells from control mice also co-expressed F4/80 (data not shown) and a much smaller portion of those cells expressed the MHC class II molecule and marker of dendritic cells CD11c. FIG. 3B shows that the treatment of tumor-bearing mice with Ad-PGDH resulted in increased expression of the MHC class II molecule by intra-tumoral F4/80-positive (left panel) and myeloid CD11b cells (right panel).

Interestingly, when tumor-infiltrated CD11b cells were isolated from the 15-PGDH-treated mice and cultured for 24 hours, the majority of these cells became double positive CD11c/F4/80 cells. Under similar conditions, tumor-infiltrated CD11b cells from control tumor-bearing mice produced significantly fewer CD11c-positive cells (Data not shown). Together, the obtained results indicate that conditioning the tumor microenvironment with the 15-PGDH gene improves differentiation of intra-tumoral myeloid antigen-presenting cells.

### Example 4

### AD-PGDH Administration Attenuates the Immunosuppressive Characteristics of Tumor-Infiltrated Myeloid Cells

The tumor-infiltrated CD11b cell population consisting of myeloid-derived suppressor cell (MDSC) and tumor associated macrophages (TAM) represents a major mediator in tumor-induced immune suppression. Tumor progression affects myelopoiesis inhibiting APC differentiation and promoting accumulation of immunosuppressive cells, which in turn inhibits the generation of adaptive anti-tumor immune responses and promotes tumor evasion (30, 31). Recent publications suggest that tumors may promote MDSC-mediated immune suppression through overproduction of PGE₂ (10, 11). Here we evaluated whether introduction of the 15-PGDH gene, which is directly involved in metabolism of PGE₂, could attenuate tumor-induced immune suppression mediated by intra-tumoral CD11b cells. Accordingly, we isolated intra-tumoral CD11b cells from treated or control tumor-bearing mice and then analyzed these cells for the ability to secrete IL-13 (FIG. 4A), measured arginase I and II expression (FIG. 4B), arginase activity (FIG. 4C), as well for activity of STATE (FIG. 4D). Obtained results indicate that the conditioning of tumor microenvironment with the 15-PGDH gene resulted in the significant 10-fold inhibition of immunosuppressive cytokine IL-13 production by intra-tumoral CD11b cells. These cells also had down-regulated arginase expression and activity. In the line with these findings, we observed reduced phosphorylation of STAT6. Since COX-2 is a major enzyme responsible for PGE₂ production, we also measured its expression in the same cells and found that PGDH-mediated treatment did not affect expression of Cox-2 (data not shown). In aggregate, our results demonstrate that intra-tumoral delivery of 15-PGDH promotes significant changes in immunosuppressive tumor microenvironment, including the strong inhibition of secretion of immunosuppressive cytokines IL-13 and IL-10, reduction of arginase expression/activity and weakening of STAT6 signaling in tumor-recruited CD11b cells.

### Example 5

### Combination of PGDH Therapy and Cancer Immunotherapy Results in Complete Tumor Rejection

To test whether 15-PGDH-mediated therapy could improve therapeutic effect of dendritic cell-based cancer immunotherapy of pre-established tumors, we used the same tumor model: CT-26 murine colon carcinoma. Tumors were established by x.c. injections of 5×10⁵ CT-26 tumor cells in to naive BALB/c mices. On day 7 after tumor injection, when tumors reached 16-25 mm² in diameter, mice were randomly divided in four groups (five mice in each group): 1) Untreated, 2) DCs alone+control Ad, (3) Ad-PGDH alone, and 4) AdPGDH+DCs. Mice were intra-tumorally injected with 1×10⁵ TCID₅₀ of adenovirus encoding 15-PGDH (Ad-PGDH) or control adenovirus encoding luciferase gene (control Ad) twice a week: on days 10, 14, 17 and 20). To assess the treatment outcome, animals were studied for their long-term survival (FIG. 5A). All untreated tumor-bearing animals died between day 25 and day 37 after tumor inoculation (0% survival). Treatment with DC resulted in complete tumor eradication and long-term surviving in 20% of mice treated with DC. In ADPGDH and ADPGDH/DC groups, 80% and 100% of mice were alive after 70 days. FIG. 5B IFN-gamma by lymph node cells in response in vitro with irradiated CT-26 or 4T1 murine tumors. Concentration of cytokines was determined using Multiplex assay. Experimental design was similar to described above in FIG. 2B. Average±SD are shown.

### Example 6

### In Vivo flt1 Promoter Activity

15-PGDH gene expression in this construct is driven by the flt1 promoter. Flt-1, a receptor for vascular endothelial growth factor (VEGFR1), is known to display a high expression in endothelial cells and tumor cells, as well as in CD11b myeloid cells. In order to evaluate the distribution of the 15-PGDH gene after adenovirus-mediated delivery, we intra-tumorally injected CT-26 tumor-bearing mice with the adenovirus encoding Renilla luciferase gene under the flt1 promoter (Ad-Luc). Forty-eight hours later, mice were sacrificed, and promoter-specific luciferase activity was determined in the tumor cell population, in isolated tumor-infiltrated CD11b cells and in CD11b-negative tumor cell population. FIG. 7 shows that the highest flt1 promoter activity was observed in the CD11b-negative tumor cell population; whereas, intra-tumoral CD11b cells demonstrated intermediate flt1 promoter activity which was lower than tumor cells but significantly higher than control levels. These data suggest that adenoviral-mediated therapy with the 15-PGDH gene under the flt1 promoter could target both tumor cells and non-tumor CD11b myeloid cells.

### Example 7

### Introduction of the 15-PGDH Gene in Tumor Tissue Inhibit PGE2 Production and Promotes Significant Changes in the Cytokine Profile of Intra-Tumoral CD11b Cells

PGE₂ overproduction has a major impact on both intra-tumoral immune and inflammatory cells favoring Th₂ cytokine milieu, inhibiting APC differentiation and promoting an immunosuppressive microenvironment. Since the 15-PGDH enzyme is known to biologically inactivate PGE₂, we examined whether introduction of the 15-PGDH gene in tumor tissue may have an impact on the PGE2 and cytokine secretion by myeloid cells. To address this hypothesis, we isolated intra-tumoral CD11b cells from treated or control tumor-bearing mice. Cells were cultured for 24 hours, then cell supernatants were collected and assayed for PGE₂ and cytokines. FIG. 8A shows purity (98%) of freshly isolated intra-tumoral CD11b cell population in one representative experiment. In separate experiments, we conducted cytologic analysis of the purified tumor-infiltrated CD11b cells, in which cytospines with CD11b-positive cells were prepared and stained with hematoxylin and eosin (data not shown). Analysis revealed that CD11b cells infiltrated CT-26 colon carcinoma consist of mostly “monocyte-macrophage” type cells with one large (non-segmented nucleus). As shown in FIG. 8B, adenoviral-mediated delivery of the 15-PGDH gene promoted the 4-fold inhibition production of PGE₂ by tumor-associated CD11b cells. Importantly, introduction of the 15-PGDH gene did not affect significantly the expression of COX-2 or mPGES1 in Ad-PGDH treated CD11b cells (FIG. 8c). Obtained data suggests that reduced PGE₂ secretion by intra-tumoral CD11b cells from treated mice is due to enhanced 15-PGDH-mediated catabolism.

### Example 8

### Expression of the 15-PGDH Gene in Colon Tumor Tissue Inhibits IL-10 and Stimulates IL-12 Cytokine Production in Draining Lymph Nodes

To examine the effect of 15-PGDH gene delivery on cytokine production in draining lymph nodes, we isolated those lymph nodes from PGDH-treated or control tumor bearing mice, prepared single suspensions and stimulated with LPS. After 24 hours of incubation, cell supernatants were collected and assayed for cytokine production. In addition, lymph nodes were analyzed by flow cytometry for intracellular cytokine production. As shown in FIG. 9, adenoviral-mediated delivery of the 15-PGDH gene induced a switch in Th₁/Th₂ cytokine expression specifically by myeloid cells. This treatment inhibited expression of Th₂ cytokine IL-10, but stimulated Th₁ cytokine IL-12 (FIG. 9A). This was associated with up-regulation in production of eotaxin and RANTES (FIG. 9B) as well as IFN-gamma, G-CSF and KC (data not shown). Importantly, we observed a similar change in Th₁/Th₂ cytokine expression in both LPS-stimulated and non-stimulated lymph node-derived CD 11b cells (data not shown).

### Example 9

### Discussion of Examples 1-8

The data shown herein demonstrates that the introduction of the PGDH gene into the tumor microenvironment results in the substantial growth inhibition of pre-established tumors in mice. The PGDH-mediated anti-tumor effect was associated with a significantly reduced secretion of immunosuppressive mediators and cytokines such as PGE₂, IL-10, IL-6 and IL-13 by intra-tumoral CD11b cell. It was also shown that expression of the 15-PGDH in the tumor promotes the in situ differentiation of M1-oriented CD11c⁺MHC class II-positive myeloid antigen-presenting cells from intra-tumoral CD11b cells, and at the same time reduces the number of immunosuppressive M2-polarized F4/80⁺ tumor-associated macrophages. Overall, the results suggest that enforced expression of the 15-PGDH gene in the tumor site can help to re-model the immunosuppressive tumor environment and promote activation of the local immune system.

In reviewing the detailed disclosure which follows, and the specification more generally, it should be borne in mind that all patents, patent applications, patent publications, technical publications, scientific publications, and other references referenced herein are hereby incorporated by reference in this application in order to more fully describe the state of the art to which the present invention pertains.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

While a number of embodiments of the present invention have been shown and described herein in the present context, such embodiments are provided by way of example only, and not of limitation. Numerous variations, changes and substitutions will occur to those of skilled in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

What is claimed is:

1. A method of correcting tumor-induced immune dysfunction and boosting a therapeutic effect of cancer immunotherapy in a subject in need thereof, said method comprising administering a polynucleotide encoding SEQ ID NO. 2 or 4, or a variant thereof comprising at least 85 percent identity to SEQ ID NO. 2 or 4, to said subject such that said polynucleotide is expressed in tumor cells of said subject; and coadministering dendritic cells to said subject, wherein said dendritic cells are activated against a tumor antigen.

2. The method of claim 1, wherein said administering comprises injecting a viral vector comprising said polynucleotide to said subject.

3. The method of claim 2, wherein said viral vector is injected into a tumor in said subject or proximate to said tumor such that said viral vector is contacted with cells of said tumor.

4. The method of claim 2, wherein said viral vector is an adenoviral vector.

5. The method of claim 1, wherein said administering comprising injecting a DNA plasmid comprising said polynucleotide into said subject.

6. The method of claim 1, wherein said tumor antigen is specific to a breast cancer, lung cancer, prostate cancer, colon cancer, rectal cancer, hepatic carcinoma, urogenital cancer, ovarian cancer, testicular carcinoma, osteosarcoma, chondrosarcoma, gastric cancer, pancreatic cancer, nasopharyngeal cancer, thyroid cancer, neuroblastoma, astrocytoma, glioblastoma multiforme, melanoma, hemangiosarcoma, an epithelial cancer, a non-epithelial cancer such as squamous cell carcinoma, leukemia, lymphoma, or cervical cancer.

7. The method of claim 1, wherein said method suppresses growth of a tumor.

8. The method of claim 7, wherein said tumor is in the colon, breast or lung of the subject.


SEQUENCE LISTING (4 sequences)

SEQ ID NO: 1 - DNA, 2550 bp, Homo sapiens
ggctgcaccatgcacgtgaacggcaaagtggcgctggtgaccggcgcggctcagggcataggcagagcctttgcagaggcgctgctgcttaagggcgccaaggtagcgctggtggattggaatcttgaagcaggtgtacagtgtaaagctgccctggatgagcagtttgaacctcagaagactctgttcatccagtgcgatgtggctgaccagcaacaactgagagacacttttagaaaagttgtagaccactttggaagactggacattttggtcaataatgctggagtgaataatgagaaaaactgggaaaaaactctgcaaattaatttggtttctgttatcagtggaacctatcttggtttggattacatgagtaagcaaaatggaggtgaaggcggcatcattatcaatatgtcatctttagcaggactcatgcccgttgcacagcagccggtttattgtgcttcaaagcatggcatagttggattcacacgctcagcagcgttggctgctaatcttatgaacagtggtgtgagactgaatgccatttgtccaggctttgttaacacagccatccttgaatcaattgaaaaagaagaaaacatgggacaatatatagaatataaggatcatatcaaggatatgattaaatactatggaattttggacccaccattgattgccaatggattgataacactcattgaagatgatgctttaaatggtgctattatgaagatcacaacttctaagggaattcattttcaagactatgatacaactccatttcaagcaaaaacccaatgaacagcttatgtgttagccatagctgaaaataagcacaaatagcttatattcagatcctatcttcatttgaatatagcttttaaatgaaatgttacagtttgaagttttccttcatgcacttggtgataaacgttttctaaatttttagttaagtatatggataaaaagttatgaactattaaaaatgtgatgtggaccaaaggctaggttgtaatcttgatagtctaaaaaatgatcaaaacaaatgattttcaaggaatattcaatattctgcctttcagaaagtgtatttatatctgtgcttcataaatattaatgttcttcagaacatcattttaaaggagatacttgaattgttatttaaatcaaaccagatgtaaaacactcacatacaagttcatactttaaaagaggaaagctacttaacaatgacaaatatttcacaataataatttttacttatataccatctttcaactgaacatttcagttcttccaagagcttcttagagtagtatattttgggggcagtcaaggaataaactacagtgtaaacatatcccagatgaaaactgctgtatggaaaaatgacagaaagtaactgattgacactgttgattcacagttcagcctcctatctgggaaagacatttctttcctctgctcactttaagaacttttaccgactccaaaaatctcaggaattaaacttttaacagttacagcaataaagaatagttagtactccaaaaatattatatttaagatgctcaacaagaaaaaaatgcaaatgtaatatttttttcaaattacttctttattgacttgtccaaatttcaaaagtgcctacccttcaataaaacttttttattctgatctccataaattacttagtcttctatgtatagctatcaaggaaataaaaccaattttgccacagccacaactgtaaatgtttttgtacccatgctgaaactcataacaacacagacataaaaatagctgtgaggttttgctttttttgttgtcagctatcttaagaatcattaaatacacctgctttgggtaaaactctttgcaagcagtaattaacactagtaacagtgaaagcacaagatttccaaatcagtcgttttctcaaaaaaatatcgtataagtgactcatcctgtctgctaactccagacctcccagcttgaagccaaatctttccatgtgagattgatatggatttcctagaagtactggaatgttgtcatatcttgccctattttaattctgctatagaaaacaattgccttcacttttaaggagtaatttgaatattaataactctggtctagattttcatataatgtattaaagacaaagtagtgaacatcaatgaacatctgatagagataaactgtaatcaggcataagcttgtttgtatgttctggcagtgactaatcagtaaatgatgtcggtttgcccagtatcacttatcttctgtatttttcctctgtcgtgtaaatagtataaccttttcatttatggacaattttttggactagtagccttcaatatacattctgctttgaattaattttttcaaatcaataaattatgtagacatttaaaatcaaatatcaagtagaattgaaaaatgtgagttacataagttaaaaacttactttaaatcttaccttctataggtagctctaaataaattcatatggttatatggcaaaaaaaaaaaaaa

SEQ ID NO: 2 - protein, 266 aa, Homo sapiens [sequence omitted]

SEQ ID NO: 3 - DNA, 2623 bp, Homo sapiens
agaacgctcagggggcaggtgacacagtcgtgggttccccggcgggcgctggcttgacagtttcctccccgcccactggcaggggagcgccccgccgggctgcacgcgcgcgcgcgcaggggggcataaaagccgcggccgcgcggagacgcggagctcgcccaccgcccgccccagcagtggctgcaccatgcacgtgaacggcaaagtggcgctggtgaccggcgcggctcagggcataggcagagcctttgcagaggcgctgctgcttaagggcgccaaggtagcgctggtggattggaatcttgaagcaggtgtacagtgtaaagctgccctggatgagcagtttgaacctcagaagactctgttcatccagtgcgatgtggctgaccagcaacaactgagagacacttttagaaaagttgtagaccactttggaagactggacattttggtcaataatgctggagtgaataatgagaaaaactgggaaaaaactctgcaaattaatttggtttctgttatcagtggaacctatcttggtttggattacatgagtaagcaaaatggaggtgaaggcggcatcattatcaatatgtcatctttagcaggactcatgcccgttgcacagcagccggtttattgtgcttcaaagcatggcatagttggattcacacgctcagcagcgcccaccattgattgccaatggattgataacactcattgaagatgatgctttaaatggtgctattatgaagatcacaacttctaagggaattcattttcaagactatgatacaactccatttcaagcaaaaacccaatgaacagcttatgtgttagccatagctgaaaataagcacaaatagcttatattcagatcctatcttcatttgaatatagcttttaaatgaaatgttacagtttgaagttttccttcatgcacttggtgataaacgttttctaaatttttagttaagtatatggataaaaagttatgaactattaaaaatgtgatgtggaccaaaggctaggttgtaatcttgatagtctaaaaaatgatcaaaacaaatgattttcaaggaatattcaatattctgcctttcagaaagtgtatttatatctgtgcttcataaatattaatgttcttcagaacatcattttaaaggagatacttgaattgttatttaaatcaaaccagatgtaaaacactcacatacaagttcatactttaaaagaggaaagctacttaacaatgacaaatatttcacaataataatttttacttatataccatctttcaactgaacatttcagttcttccaagagcttcttagagtagtatattttgggggcagtcaaggaataaactacagtgtaaacatatcccagatgaaaactgctgtatggaaaaatgacagaaagtaactgattgacactgttgattcacagttcagcctcctatctgggaaagacatttctttcctctgctcactttaagaacttttaccgactccaaaaatctcaggaattaaacttttaacagttacagcaataaagaatagttagtactccaaaaatattatatttaagatgctcaacaagaaaaaaatgcaaatgtaatatttttttcaaattacttctttattgacttgtccaaatttcaaaagtgcctacccttcaataaaacttttttattctgatctccataaattacttagtcttctatgtatagctatcaaggaaataaaaccaattttgccacagccacaactgtaaatgtttttgtacccatgctgaaactcataacaacacagacataaaaatagctgtgaggttttgctttttttgttgtcagctatcttaagaatcattaaatacacctgctttgggtaaaactctttgcaagcagtaattaacactagtaacagtgaaagcacaagatttccaaatcagtcgttttctcaaaaaaatatcgtataagtgactcatcctgtctgctaactccagacctcccagcttgaagccaaatctttccatgtgagattgatatggatttcctagaagtactggaatgttgtcatatcttgccctattttaattctgctatagaaaacaattgccttcacttttaaggagtaatttgaatattaataactctggtctagattttcatataatgtattaaagacaaagtagtgaacatcaatgaacatctgatagagataaactgtaatcaggcataagcttgtttgtatgttctggcagtgactaatcagtaaatgatgtcggtttgcccagtatcacttatcttctgtatttttcctctgtcgtgtaaatagtataaccttttcatttatggacaattttttggactagtagccttcaatatacattctgctttgaattaattttttcaaatcaataaattatgtagacatttaaaatcaaatatcaagtagaattgaaaaatgtgagttacataagttaaaaacttactttaaatcttaccttctataggtagctctaaataaattcatatggttatatggcatctctggtgtatactgattgagaaaataattaaactgaagttaggggaggggaaaaaaaaaaaaaaaaa

SEQ ID NO: 4 - protein, 178 aa, Homo sapiens
MHVNGKVALVTGAAQGIGRAFAEALLLKGAKVALVDWNLEAGVQCKAALDEQFEPQKTLFIQCDVADQQQLRDTFRKVVDHFGRLDILVNNAGVNNEKNWEKTLQINLVSVISGTYLGLDYMSKQNGGEGGIIINMSSLAGLMPVAQQPVYCASKHGIVGFTRSAAPTIDCQWIDNTH